(19) United States
(12) Patent Application Publication
(10) Pub. No.: US 2024/0133880 A1
(43) Pub. Date: Apr. 25, 2024

(54) HOMOGENOUS ENZYME LINKED IMMUNOASSAY

(71) Applicant: IDEXX Laboratories, Inc., Westbrook, ME (US)

(72) Inventors: Jennifer Gagner, Portland, ME (US); Dheeraj Khare, Cape Elizabeth, ME (US); Daniel Wayne Patch, Windham, ME (US); Evan Michael Peck, Gorham, ME (US); James Robert Salter, Marietta, GA (US); Christopher Peter Turmel, Westbrook, ME (US)

(21) Appl. No.: 17/962,094

(22) Filed: Oct. 7, 2022

Related U.S. Application Data
(60) Provisional application No. 63/256,030, filed on Oct. 15, 2021.

(51) Int. Cl. G01N 33/558; G01N 33/543

(52) U.S. Cl. CPC G01N 33/558; G01N 33/54386

(57) ABSTRACT

The present disclosure relates to methods for determining the presence of or the amount of an analyte in a sample and to a slide for use in the method.

### CROSS-REFERENCE TO RELATED APPLICATIONS

This application claims the benefit of U.S. Provisional Application Ser. No. 63/256,030, filed on Oct. 15, 2021. The contents of which are expressly incorporated herein.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND

The technology relates to a homogeneous enzyme immunoassay that uses a slide and does not require any separation steps, such as washing steps. The technology further relates to a dry slide used in the method.

### DESCRIPTION OF RELATED ART

Immunoassay is a technique for measuring the presence or concentration of a substance in a test sample, typically a solution, that frequently contains a complex mixture of substances. Typically, the test sample is a biological fluid, such as serum or urine. Immunoassay is based on the unique ability of an antibody, or other protein, to bind with high specificity to one or a very limited group of molecules. A molecule that binds to an antibody is called an antigen. Immunoassays can be carried out to measure the presence or concentration of either the antigen or the antibody (i.e., either the antigen or the antibody can be the analyte). In either case, the specificity of the assay depends on the degree to which the analyte is able to bind to its specific binding partner to the exclusion of other substances that might be present in the sample being analyzed. In addition to the need for specificity, a binding partner must be selected that has a sufficiently high affinity for the analyte to permit an accurate measurement.

A requirement of immunoassays is a means to produce a measurable signal in response to a specific binding event. This can be accomplished by measuring a change in some physical characteristic, such as light scattering or refractive index, that occurs when the analyte is bound to its binding partner. Many immunoassays depend on the use of a binding partner that is associated with a detectable label. A binding partner associated with a detectable label is often referred to as a tracer. A large variety of detectable labels have been used, including radioactive elements (used in radioimmunoassay); enzymes; fluorescent, phosphorescent, and chemiluminescent dyes; latex and magnetic particles; dye crystallites; gold, silver, and selenium colloidal particles; metal chelates; coenzymes; electroactive groups; oligonucleotides, stable radicals, and others. Such detectable labels permit detection and quantitation of binding events either after separating free and bound tracer or by designing the system in such a way that a binding event effects a change in the signal produced by the tracer.

Immunoassays requiring a separation step, often called separation immunoassays or heterogeneous immunoassays, typically require multiple steps, for example, careful washing of a surface to separate tracer that is bound to its binding partner from unbound tracer. An immunoassays in which a signal is affected by binding that is run without a separation step is called a homogenous immunoassay. A homogenous immunoassay is carried out by simply mixing the reagents and sample and making a physical measurement. Homogenous immunoassays are easier to perform than heterogenous immunoassays.

Regardless of the method used, interpretation of the signal produced in an immunoassay requires reference to a standard that mimics the characteristics of the sample medium. For qualitative assays the standards may consist of a reference sample with no analyte and a positive sample having the lowest concentration of the analyte that is considered detectable. Quantitative assays require additional standards with known analyte concentrations. Comparison of the assay response of a test sample to the assay responses produced by the standards makes it possible to interpret the signal strength in terms of the presence or concentration of the analyte in the sample.

An immunoassay can be competitive or non-competitive. In a competitive immunoassay, the antibodies in a sample compete with a tracer (i.e., an antibody linked to a detectable label) to bind with an antigen. The amount of tracer bound to the antibody is then measured. In a competitive immunoassay, the amount of tracer bound to the antibody is inversely related to the concentration of antibodies in the sample. This is because when there are higher amounts of antibodies in the sample more antigen binds to the antibodies in the sample and less antigen is available to bind to the tracer.

In noncompetitive immunoassays, also referred to as a “sandwich assay,” antigen in the sample is bound to an antibody fixed to a surface, then a second antibody, which is attached to a detectable label, is bound to the antigen. The amount of bound detectable label is then measured. Unlike the competitive immunoassay, in the noncompetitive immunoassay the response is directly proportional to the concentration of the antigen in the sample. This is because the detectable label on the second antibody will not be bound if the antigen is not present in the sample.

These techniques are not limited to antibody-antigen binding partners. Similar assays can be performed using a protein (which is not an antibody) to determine the presence or concentration of a substrate that specifically binds to the protein. The tracer in these assays can be, for example, a detectable label linked to the protein or a detectable label linked to a molecule that binds to the protein.

Immunoassays are advantageous over other analytical methods for measuring the presence or concentration of a substance in a test sample, such as, for example, gas chromatography (GC) and high-performance liquid chromatography (HPLC), because immunoassays avoid the extractions and other complex sample work-up procedures and lengthy assay times that are often associated with these other analytical methods.

However, there remains room for improvement, e.g. immunoassays that exhibit higher sensitivity, are simpler to perform, and/or are less expensive to perform.

These and other features and advantages of the present invention will become apparent from the remainder of the disclosure, in particular the following detailed description of the preferred embodiments, all of which illustrate by way of example the principles of the invention.

Citation of any reference in this application is not to be construed that such reference is prior art to the present application.

### SUMMARY

The technology is directed to a homogeneous enzyme immunoassay that uses a slide and does not require any separation steps, such as washing steps. The technology is also directed to a slide used in the assay.

In one embodiment, the method for determining the presence of or the amount of an analyte in a sample comprises:
(i) providing a sample suspected of containing an analyte;
(ii) contacting the sample with a binding partner-enzyme conjugate attached to the surface of particles to provide a mixture comprising the binding partner-enzyme conjugate attached to the surface of particles and the sample,
   wherein analyte in the mixture can displace the binding partner-enzyme conjugate from the surface of the particles,
(iii) separating the particles from the mixture to provide a particle free mixture;
(iv) contacting the particle free mixture with a polymer comprising a substrate for the enzyme dispersed in the polymer,
   wherein interaction of the substrate and the enzyme produces a detectable signal.

In one embodiment, the method uses a slide comprising two layers. The two layers are:
(i) a first layer comprising a binding partner-enzyme conjugate attached to the surface of particles dispersed in a first polymer,
   wherein the binding partner-enzyme conjugate is specific for the analyte; and
(iii) a second layer comprising a substrate for the enzyme dispersed in a second polymer,
   wherein interaction of the substrate and the enzyme produces a detectable signal.

The method for determining the presence of or the amount of an analyte in a sample using the slide comprising two layers involves:
(i) providing a sample suspected of containing an analyte;
(ii) contacting the sample with the first layer of the slide; and
(iii) determining if there is a detectable signal.

In one embodiment, the method uses a slide comprising three layers. The three layers are:
(i) a first layer comprising a binding partner-enzyme conjugate dispersed in a first polymer, wherein the binding partner-enzyme conjugate is specific for the analyte;
(ii) a second layer comprising solid particles that have analyte bound to their surface dispersed in a second polymer, and
(iii) a third layer comprising a substrate for the enzyme dispersed in a third polymer, wherein interaction of the substrate and the enzyme produces a detectable signal.

The method for determining the presence of or the amount of an analyte in a sample using the slide comprising two layers involves:
(i) providing a sample suspected of containing an analyte;
(ii) contacting the sample with the first layer of the slide of; and
(iii) determining if there is a detectable signal.

In one embodiment, the method further comprises measuring the intensity of the detectable signal.

In one embodiment, the method does not require any separation steps.

### BRIEF DESCRIPTION OF THE DRAWINGS

FIG. 1 depicts the general structure of the slide used in the enzyme linked immunosorbent assay described herein.

FIG. 2 depicts the principal by which the enzyme linked immunosorbent assay described herein works.

FIG. 3 depicts the reading layer of a slide from a homogeneous enzyme immunoassay as described herein. FIG. 3A depicts the reading layer in an assay that used TBM as the substrate and FIG. 3B depicts the reading layer in an assay that used an ion-pair between TMB and succinic acid as the substrate.

FIG. 4A depicts the general structure of a two layer slide used in the enzyme linked immunosorbent assay described herein. FIGS. 4B and 4C depict the structure of a two layer slide further comprising an optional double-sided tape layer and an optional support layer. In FIG. 4B the read layer is Whatman 595 membrane attached to a solid PET support. In FIG. 4C the read layer is a cellulose polymer coated on a solid PET support.

FIG. 5A depicts the principal by which the method using a slide comprising (i) a layer comprising a binding partner-enzyme conjugate attached to the surface of particles and (iii) a read layer as described herein. FIG. 5B is a schematic depicting the process described in FIG. 5A.

FIG. 6 is a plot of the percent area of a read layer that is wetted when a first aqueous sample is applied to a slide having a binding partner-enzyme conjugate layer and a read layer that are separated by dissolvable double-sided tape followed by application of a second aqueous sample as a function of time, t, between application of the first aqueous sample and the second aqueous sample.

FIG. 7 is a plot of Response v. SDMA concentration when a sample containing SDMA was assayed for SDMA with a slide having an binding partner-enzyme conjugate layer a read layer.

### DETAILED DESCRIPTION

The technology encompasses a homogeneous enzyme immunoassay that uses a slide and does not require any separation steps, such as washing steps.

The technology also encompasses the slide used in the enzyme linked immunosorbent assay.

### 1. Definitions

The term “analyte,” as that term is used herein, refers to a molecule (e.g., antibody or antigen) that is present in a sample, such as a biological fluid, whose presence or concentration in the sample is intended to be determined and which binds to (i.e., forms a complex with) a binding partner (e.g., antigen or antibody).

A “complex,” as that term is used herein, is a species formed by an association of two or more molecular entities (which can be ionic or uncharged) that does not involve a covalent bond between the entities. Examples of a complex are the association of an antibody with an antigen and the association of a peptide with a receptor.

The term “hapten,” as that term is used herein, is a molecule that does not induce antibody formation when injected into an animal but can be linked to a carrier protein to provide an antigen (immunogen) that elicits an immune response when injected into an animal that results in the formation of antibodies. The resulting antibodies may be isolated by known antibody isolation techniques. The hapten binds to the resulting antibody.

The term “antigen,” as used herein, has its art recognized meaning, i.e., a substance that when introduced into the body stimulates the production of an antibody.

The term “antibody,” as used herein, has its art recognized meaning, i.e., a protein produced because of the introduction of an antigen into a body.

The phrase “binding partner,” as that phrase is used herein, means a molecule that binds a second molecule with specificity. For example, the second molecule can be an antigen/antibody and the “binding partner” can be an antibody/antigen. Similarly, the second molecule can be a peptide for a receptor and the “binding partner” can be the receptor or the second molecule can be the receptor and the “binding partner” can be the peptide. The second molecule can be an analyte.

The phrases “with specificity,” “specifically binds,” “binds the analyte with specificity,” “specific for the analyte,” and similar phrases, as used herein, have their art-recognized meaning, i.e., that the binding partner recognizes and binds to an analyte (or a class of analytes) with greater affinity than it binds to other non-specific molecules. For example, an antibody raised against an antigen that binds the antigen more efficiently than other non-specific molecules can be described as specifically binding to the antigen. Binding specificity can be tested using methodology known in the art such as, for example, an enzyme-linked immunosorbant assay (ELISA), a radioimmunoassay (MA), or a western blot assay. A non-specific molecule is a molecule that shares no common epitope with the analyte.

The phrase “epitope,” as used herein, means the portion of an analyte that has the structural, spatial, and polar arrangement so as to define one or more determinant or epitopic sites of the analyte so that the analyte is capable of being recognized by and specifically bind to its binding partner. The phrase “epitopic moiety,” as used herein, means an epitopic moiety or a molecular entity that includes the epitopic moiety

The phrase “binding partner-enzyme conjugate,” as that term is used herein, means a binding partner that is covalently bound to an enzyme that acts as a detectable label. For example, the binding partner-enzyme conjugate could be an antibody that is specific for an antigen, wherein the antibody is conjugated to (i.e., covalently bound to) an enzyme that acts as a detectable label. The antigen has a structural, spatial, and polar arrangement of atoms that define one or more determinant or epitopic sites (i.e., an “epitopic moiety”) that is/are recognized by the antibody so that the antibody will specifically bind to the antigen.

The phrase “detectable label,” as used herein, means the part of a binding partner-enzyme conjugate that permits detection and quantitation of the binding partner-enzyme conjugate. For example, an enzyme, which can produce a color change when contacted with a substrate for the enzyme, that is covalently bound to a binding partner (e.g., an antibody) is the “detectable label” of the binding partner-enzyme conjugate that comprises the antibody and the enzyme.

The binding partner-enzyme conjugate can be an antibody conjugated to an enzyme. The binding partner-enzyme conjugate can be a receptor for a peptide conjugated to an enzyme. The binding partner-enzyme conjugate can be an antigen conjugated to an enzyme. The binding partner-enzyme conjugate can be a peptide specific for a receptor conjugated to an enzyme.

The phrase “substantially free of,” as used herein means less than 10%, preferably less than 5%, more preferably less than 2%, and most preferably less than 1%.

The phrase “proportional to,” as used herein, means that there is a correspondence or relationship between a measured value and the amount of something. For example, the phrase “the intensity of the signal is directly proportional to the concentration of the antigen in the sample” means that the intensity of a signal corresponds to the concentration of an antigen in a sample. The measured value and the amount may be linearly related.

The term “about,” as used herein means±10%, preferably ±5%, more preferably, ±2%, and most preferably ±1%.

### 2. Description of the Method

The technology is directed to methods for determining the presence of or the amount of an analyte in a sample. The technology is also directed to a slide used in the assay.

### 1. Assay Method Using a Slide Comprising a Read Layer

In one embodiment the method for determining the presence of or the amount of an analyte in a sample comprises the steps of:
(i) providing a sample suspected of containing an analyte;
(ii) contacting the sample with a binding partner-enzyme conjugate attached to the surface of particles to provide a mixture comprising the binding partner-enzyme conjugate attached to the surface of particles and the sample;
   wherein analyte in the mixture can displace the binding partner-enzyme conjugate from the surface of the particles,
(iii) separating the particles from the mixture to provide a particle free mixture;
(iv) contacting the particle free mixture with a polymer comprising a substrate for the enzyme dispersed in the polymer,
   wherein interaction of the substrate and the enzyme produces a detectable signal.

The polymer comprising a substrate for the enzyme dispersed in the polymer is referred to herein as the read layer. The read layer comprises a substrate for an enzyme that produces a detectable signal (such as a color change) when the enzyme interacts with the substrate. In this embodiment, all reactions, except for the detection step, can be performed off the slide in a reaction vessel.

In this embodiment, a binding partner-enzyme conjugate is incubated with particles having analyte attached to their surface. The binding partner-enzyme conjugate forms a complex with analyte on the surface of the particles to provide particles having the binding partner-enzyme conjugate attached to their surface. A sample suspected of containing an analyte whose presence or amount is to be determined is combined with the particles having the binding partner-enzyme conjugate attached to their surface. Analyte in the sample competes with analyte bound to the surface of the particles to form a complex of the analyte with the binding partner-enzyme conjugate (i.e., the binding partner-enzyme conjugate binds to the analyte and is released from the particles). An aliquot of the resulting solution is then removed, without removing the particles, and applied to the read layer. The enzyme interacts with the substrate in the read layer to form a detectable signal (e.g., a color change).

When the complex of the analyte and binding partner-enzyme conjugate, separated from the particles, is applied to the read layer, the binding partner-enzyme conjugate catalyzes a reaction with the substrate to provide a signal, such as development of a color, that is measured. The signal is measured in the read layer. The intensity of the signal is directly proportional to the concentration of the analyte in the sample, i.e., the higher the analyte concentration in the sample, the more intense the signal.

The sample and the binding partner-enzyme conjugate attached to the surface of particles are typically contacted in a liquid medium. In one embodiment, the liquid medium is an aqueous medium.

There are numerous methods for conjugating binding partners to enzymes to provide a binding partner-enzyme conjugate.

In one embodiment, the method for conjugating the binding partner to the enzyme involves forming sulfhydryl groups on the binding partner (which can be an antibody) by reducing cystine groups on the binding partner. In another embodiment, the binding partner is contacted with N-succinimidyl S-acetylthioacetate (SATA) to add sulfhydryl-containing group to a primary amine on the binding partner. The binding partner is then contacted with the enzyme. In one embodiment, the molar ratio of the enzyme to the binding partner ranges from about 1:1 to about 20:1. In one embodiment, the molar ratio of the enzyme to the binding partner ranges from about 4:1 to about 12:1.

Other methods for conjugating the binding partner to the enzyme also exist. When the binding partner is an antibody, methods of conjugation include, e.g., SMCC (succinimidyl 4-(N-maleimidomethyl)cyclohexane-1-carboxylate) chemistry, SPDP (succinimidyl 3-(2-pyridyldithio)propionate) chemistry, periodate chemistry, and tetrazine click chemistry. Kits containing reagents in instructions for carrying out each of these methods are commercially, for example from ThermoFisher Scientific.

In one embodiment, the binding partner is an antibody. In other words, the binding partner-enzyme conjugate layer is an antibody-enzyme conjugate layer. The antibody-enzyme conjugate layer comprises an antibody, which is specific for an antigen, that has been conjugated to an enzyme, which acts as a detectable label, so as to provide an antibody-enzyme conjugate.

Antibodies can be obtained by developing an immune response in an animal to the antigen using art recognized techniques. Typically, a hapten (which has an epitopic moiety in common with the analyte of interest) is conjugated to a carrier protein, such as bovine serum albumin, to provide an immunogen (antigen) that is administered to an animal, such as a rabbit, mouse, or sheep, by a series of injections and then the resulting antibodies isolated using conventional techniques. Other illustrative protein carriers that can be used to form the immunogen include, but are not limited to, keyhole limpet hemocyanin, egg ovalbumin, bovine gamma-globulin, and thyroxine-binding globulin. Alternatively, the antigen can be formed by conjugating the hapten to a synthetic or natural polymeric material that contains a functional group that is reactive with the hapten.

The binding partner-enzyme conjugate is attached to the surface of the particles by first forming particles having analyte attached to their surface and then combining the particles having analyte attached to their surface with the binding partner-enzyme conjugate.

Particles to which the analyte can be attached include, but are not limited to, latex particles. In one embodiment, the particles are latex particles of about 0.5 μm to about 5.0 μm passively coated with the analyte. In one embodiment, the particles are latex particles about 1.0 μm to about 2.0 μm passively coated with the analyte. Latex beads of various sizes can be purchased from ThermoFisher Scientific, Waltham, MA, USA.

In one embodiment, the analyte is attached to the surface of the particles via a protein. In some embodiments, the protein is G6PDH. In some embodiments, the analyte is first conjugated to G6PDH, and the analyte-G6PDH conjugate is non-covalently attached (coated) to the surface of the particles. In particular embodiments, the analyte is MMA. Procedures for the conjugation of MMA to G6PDH and the coating of G6PDH-MMA conjugate onto particles is described in co-pending U.S. Pat. No. 11,422,136.

An embodiment t encompasses the solid particles with analyte attached to their surface.

An embodiment encompasses the solid particles with the binding partner-enzyme conjugate attached to the surface of the particles.

The read layer comprises a substrate for an enzyme that produces a detectable signal (such as a color change) when the enzyme interacts with the substrate. The substrate for the enzyme is dispersed in a polymer to provide the read layer.

Suitable polymers for the read layer include, but are not limited to, celluloses.

Typically, the concentration of the substrate for the enzyme in the read layer ranges from 0.1% to about 2.0%.

In one embodiment, the thickness of the read layer ranges from about 25 μm to about 300 μm. In one embodiment, the thickness of the read layer ranges from about 50 μm to about 250 μm. In one embodiment, the thickness of the read layer ranges from about 100 μm to about 200 μm. In one embodiment, the thickness of the read layer is about 150 μm. The thickness of the read layer depends in part on the sample volume. For example, when the sample is a very dilute solution of the analyte, the method will require a larger sample volume and a thicker read layer will be preferred so as to accommodate the larger sample volume.

The read layer can be prepared by dissolving/suspending the substrate in a solvent and applying the resulting solution/suspension to a preformed sheet of the polymer and then drying the wetted polymer. In one embodiment, the solution/suspension is an aqueous solution/suspension. The concentration of the substrate in the solution/suspension ranges from about 0.05% to about 1.0%. In one embodiment, the read layer is prepared by dipping the preformed sheet of the polymer in the solution/suspension of the substrate or by applying a solution/suspension of the substrate to the polymer and then drying the polymer, e.g., about 45° C. for about 10 minutes to about 15 minutes. In one embodiment, the preformed sheet of polymer is a Whatman 595 filtration membrane (commercially available from GE Life Sciences, Rahway, NJ). The Whatman 595 filtration membrane is a cellulose membrane.

In one embodiment, the preformed sheet of polymer (e.g., Whatman 595 or Whatman 6 or Whatman 50 filtration membrane) is attached to a clear support layer, such as a polyethylene terephthalate (PET) layer. Sheets of other optically translucent materials may also be used as the support layer. A pressure sensitive adhesive can be used to attach the preformed sheet of polymer to the solid support layer.

In one embodiment, the read layer is prepared by coating a clear support layer with a solution/suspension comprising the substrate and the polymer (e.g., cellulose) and then removing solvent from the solution/suspension. The components of the read layer are combined in a solvent with mixing to provide a solution/suspension, the resulting solution/suspension is applied to the clear support layer, and the solvent removed so as to provide the read layer on top of the clear support layer. Any method of coating can be used to provide each layer of the slide at the desired thickness. Suitable coating techniques are described in “Liquid Film Coating: Scientific principles and their technological implications,” Ed. Stephan F. Kistler and Peter M. Schweizer, First Ed., 01997 Springer Science+Business Media Dordrecht. In one embodiment, the read layer is applied using a knife coater. In one embodiment, the read layer is applied using a loop coater.

In one embodiment, the support layer is polyethylene terephthalate (PET). Typically, the thickness of the support layer ranges from about 15 μm to about 150 μm. In one embodiment, the thickness of the support layer ranges from about 25 μm to about 125 μm. In one embodiment, the thickness of the support layer ranges from about 50 μm to about 100 μm. In one embodiment, the thickness of the support layer is about 75 μm.

In one embodiment, the solution/suspension for providing the read layer is prepared by combining the following components in 90% ethanol: succinic acid at a concentration of about 0.1 to about 1.0 percent by weight, preferably about 0.8 percent by weight; substrate for the enzyme (e.g., 3,3′,5,5′-tetramethylbenzidine (TMB), when the enzyme is a laccase) at a concentration of about 0.1 to about 0.2 percent by weight, preferably about 0.13 percent by weight; D4 hydrogel solution (commercially available from AdvanSource Biomaterials Corp of Wilmington, MA) at a concentration of about 0.9 to about 5 percent by weight, preferably about 3 percent by weight; cellulose at a concentration of about 24 to about 35 percent by weight, preferably about 27 percent by weight; and optionally hydroxypropyl cellulose (HPC) at a concentration of about 0 to about 3 percent by weight. The pH of the resulting solution/suspension ranges from about 3.5 to about 4.0.

In one embodiment, the solution/suspension for providing the read layer is prepared by combining about 116.4 mg succinic acid in about 6.08 g of 90% ethanol, adding about 19.7 mg of 3,3′,5,5′-tetramethylbenzidine (TMB) with mixing, adding about 4.93 g of a 9.13% solution of D4 polyurethane with mixing, and adding about 4.09 g cellulose with mixing. The pH of the resulting solution/suspension ranges from about 3.5 to about 4.0.

The resulting solution/suspension is then coated on a support layer and the solution/suspension dried. The thickness of the wet read layer typically ranges from about 310 μm to about 320 μm. The thickness of the dry read layer typically ranges from about 155 μm to about 160 μm.

Illustrative enzymes include, but are not limited to, a laccase, a beta-galactosidase, a beta-lactamase, and a luciferase. In one embodiment, the enzyme is a laccase. Lacasses occur in a variety of fungal and bacterial species, including Myceliophthora thermophila, Thermothelomyces thermophila, Botrytis aclada, Streptomyces cyanes, and Thermus thermophilus. An illustrative lacasse is lacasse purified from Thermothelomyces thermophila commercially available from Sigma Aldrich, St. Louis, MO, which can be purified using a conA sepharose column.

In one embodiment, the lacasse is a laccase that has been recombinantly expressed (e.g., cloned laccase genes from Thermothelomyces thermophila, Botrytis aclada, Aspergillus niger, Streptomyces cyaneus, Thermus thermophilus). In one embodiment, a DNA sequence encoding a laccase from Thermothelomyces thermophila (NCBI Reference Sequence XP 003663741.1), from Botrytis aclada (UniProt accession H8ZRU2), from Aspergillus niger (NCBI Reference Sequence XP 001392958.1), from Streptomyces cyaneus (GenBank accession ADX97492.1), or from Thermus thermophilus (Uniprot accession Q72HW2) is cloned onto a pET28a vector and expressed in E coli. In one embodiment, a DNA sequence encoding a laccase from Myceliophthora thermophila (Genbank accession AAE33170.1) is cloned into a vector suitable for expression in Pichia pastoris. The lacasses from Thermothelomyces thermophila, Botrytis aclada, Aspergillus niger, and Myceliophthora thermophila are fungal lacasses and the lacasses from Streptomyces cyaneus and Thermus thermophilus are bacterial lacasses.

In one embodiment, the enzyme is a non-mammalian enzyme. Employing a non-mammalian enzyme advantageously avoids potential interference from compounds present in a mammalian sample that would be a substrate for a mammalian enzyme but are not a substrate for the non-mammalian enzyme. In a preferred embodiment, the sample is obtained from a mammal and the enzyme is a non-mammalian enzyme.

In a preferred embodiment, the non-mammalian enzyme is Fungal Laccase and the substrate is TMB. Fungal Laccase reacts with TMB to form a blue color. The intensity of the blue color is proportional to the binding partner-enzyme conjugate applied to the read layer, which is directly proportional to the amount of analyte in the sample. Fungal Laccase also advantageously catalyzes the oxidation of TMB using oxygen present in the air and, thus, avoids having to use hydrogen peroxide as a co-factor for the enzymatic reaction.

In one embodiment, the analyte is an antigen; the binding partner-enzyme conjugate is an antibody-enzyme conjugate, wherein the antibody is specific for the antigen; and the enzyme is a non-mammalian enzyme. In a preferred embodiment, the analyte is an antigen; the binding partner-enzyme conjugate is an antibody-enzyme conjugate, wherein the antibody is specific for the antigen; and the enzyme is Fungal Laccase.

In a particularly preferred embodiment, the non-mammalian enzyme is Fungal Laccase and the substrate is an ion-pair formed between TMB and a divalent acid, such as succinic acid. The structure of the ion-pair can be:

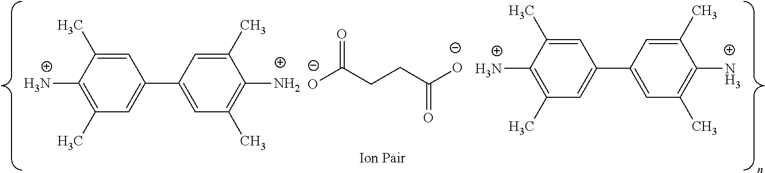

wherein n is an integer.

Suitable divalent acids include, but are not limited to, succinic acid, oxalic acid, malonic acid, glutamic acid, adipic acid, and pimelic acid. Succinic acid is a preferred divalent acid.

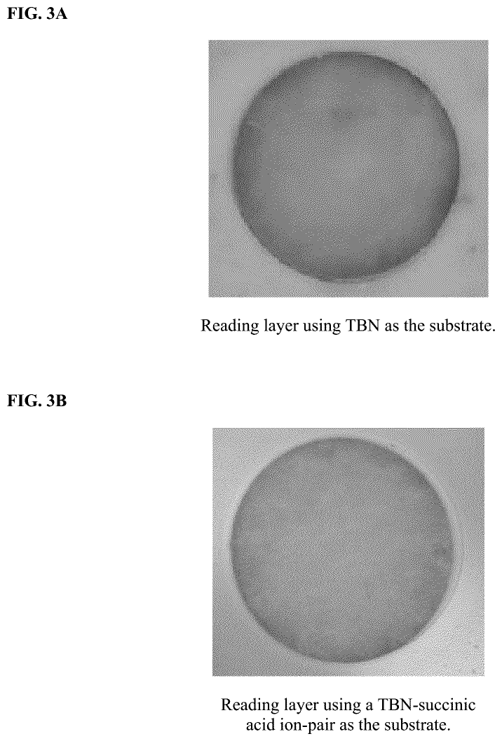

Using an ion-pair formed between TMB and a divalent acid, such as succinic acid, as the substrate advantageously avoids the blue color provided from reaction of the enzyme with the TMB in the read layer from exhibiting the “coffee ring” effect, wherein a more intense color is observed at the periphery of the read layer compared to the center of the read layer. Thus, the blue color is more evenly dispersed in the read layer when an ion-pair formed between TMB and a divalent acid, such as succinic acid, is used as the substrate compared to TMB alone being used as the substrate. FIG. 3A depicts the reading layer in an assay that used TBM as the substrate and FIG. 3B depicts the reading layer in an assay that used an ion-pair between TMB and succinic acid as the substrate. The assay used to provide FIG. 3 involved SDMA as the antigen and an antibody-enzyme conjugate, wherein the antibody was an antibody to SDMA, and the enzyme was Fungal Laccase. Antibodies to SDMA are described in, for example, U.S. Pat. No. 8,481,690.

In one embodiment, the read layer further comprises an activator to increase the activity of the laccase. Illustrative activators include, but are not limited to, 2,2′-azino-bis(3-ethylbenzothiazoline-6-sulfonic acid (ABTS); 3,5-dimethoxy-4-hydroxyacetophenone; ferulic acid; and p-coumaric acid.

In one embodiment, the read layer further comprises a mediator to increase the signal from the enzymatic reaction. Illustrative mediators include, but are not limited to 1-hydroxybenzotriazole (HBT), 2-hydroxyisoindoline-1,3-dione (HPI); N-hydroxy-N-phenylacetamide (NHA); (2,2,6,6-Tetramethylpiperidin-1-yl)oxyl (TEMPO); and violuric acid.

In one embodiment, wherein the substrate is TMB, the read layer is prepared by dipping a Whatman 595 cellulose membrane into a solution of 1000 ug/ml TMB in 100 mM succinic acid at a pH of equal to or less than about 2.5 and then drying the membrane at about 45° C. for about 10 minutes to about 15 minutes. The TMB-succinic acid solution can be prepared by either of two procedures.
Procedure 1: (i) Provide the desired volume of TMB buffer (TMB concentration 400 ug/ml) (commercially available from Thermo Scientific, Waltham, MA), (ii) Per each mL of Thermo TMB buffer, add 600 ug of solid TMB (commercially available from Sigma Aldrich, St. Louis, MO), and (iii) Per each mL of the resulting solution add 11.8 mg of succinic acid to achieve a final molarity of 100 mM succinic acid and 1000 ug/ml TMB.
Procedure 2: (i) Add 1200 uL of 0.5M succinic acid to 6 mg of TMB powder, (ii) Add 4 mL of deionized water, (iii) Add about 30 μL of 1N HCl to adjust the pH of the TMB solution to about 2.4, and (iv) bring the solution to a final volume of about 6 mL with deionized water to achieve a final molarity of 100 mM succinic acid and 1000 ug/ml TMB.

In one embodiment, the slide further comprises one or more of a spreading layer, a reflective layer, and a carbon black-containing layer, as described below.

### 2. Assay Method Using a Slide Comprising (i) a Layer Comprising a Binding Partner-Enzyme Conjugate Attached to the Surface of Particles and (ii) a Read Layer

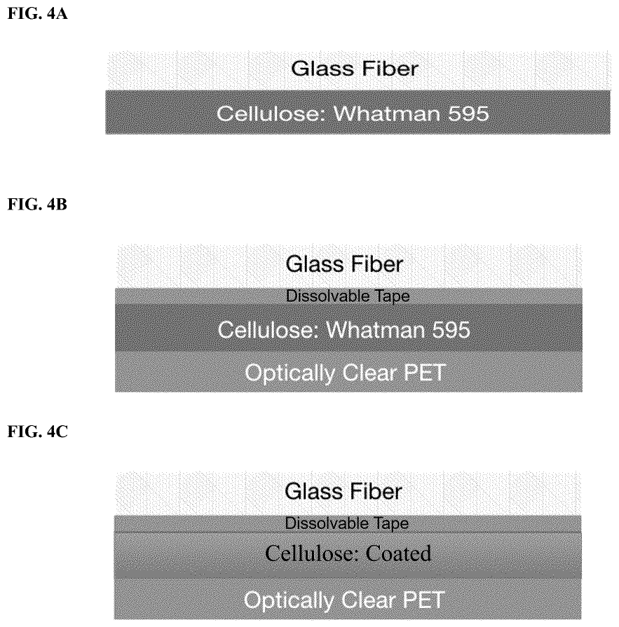

In one embodiment, the method uses a slide comprising two layers. The two layers are: (i) a layer comprising a binding partner-enzyme conjugate attached to the surface of particles and (iii) a read layer. A schematic of a slide comprising two layers is depicted in FIG. 4A. In FIG. 4A the layer comprising a binding partner-enzyme conjugate attached to the surface of particles is a glass fiber layer and the read layer is a Whatman 595 cellulose layer.

The method involves applying a sample suspected of containing an analyte to the layer comprising a binding partner-enzyme conjugate attached to the surface of particles.

In operation, a sample suspected of containing an analyte (which can be an antigen) whose presence or amount is to be determined is added to the layer comprising a binding partner (which can be an antibody)-enzyme conjugate attached to the surface of particles, analyte (e.g., antigen) in the sample forms a complex with the binding partner-enzyme conjugate present in the layer comprising a binding partner-enzyme conjugate attached to the surface of particles. The complex of the analyte and binding partner-enzyme conjugate then diffuses into the read layer wherein the enzyme on the binding partner-enzyme conjugate (which is bound to analyte) catalyzes a reaction with the substrate to provide a signal, such as development of a color, that is measured. The signal is measured in the read layer.

The intensity of the signal is directly proportional to the concentration of the analyte in the sample, i.e., the higher the analyte concentration in the sample, the more intense the signal.

The layer comprising a binding partner-enzyme conjugate attached to the surface of particles comprises a binding partner-enzyme conjugate attached to the surface of particles that are dispersed in a glass fiber membrane.

In one embodiment, the binding partner-enzyme conjugate comprises an antibody that is specific for an antigen wherein the antibody is conjugated to (i.e., covalently bound to) an enzyme that acts as a detectable label (i.e., the binding partner-enzyme conjugate layer is an antibody-enzyme conjugate layer).

Methods for forming a binding partner-enzyme conjugate are discussed above.

Methods for forming particles with the binding partner-enzyme conjugate attached to the surface of particles are described above.

### The Layer Comprising a Binding Partner-Enzyme Conjugate Attached to the Surface of Particles

The binding partner-enzyme conjugate attached to the surface of particles is dispersed in a glass fiber membrane. An illustrative glass fiber membrane suitable for forming the layer comprising a binding partner-enzyme conjugate attached to the surface of particles is a Whatman LF1 filter (commercially available from Cytiva, Marlborough, MA) having a thickness of about 330 μm.

The binding partner-enzyme conjugate attached to the surface of particles can be dispersed in the glass fiber membrane by directly spotting a solution/suspension of the binding partner-enzyme conjugate attached to the surface of particles onto the glass fiber membrane and drying the wetted membrane. In one embodiment, the solution/suspension is an aqueous solution/suspension. In one embodiment, the solution/suspension of the binding partner-enzyme conjugate attached to the surface of particles is spotted on the glass fiber membrane using a Nordson EFD dispenser (commercially available from Nordson EFD, East Providence, RI). In one embodiment, the solution/suspension of the binding partner-enzyme conjugate attached to the surface of particles is spotted on the glass fiber membrane using a solution/suspension of the binding partner-enzyme conjugate attached to the surface of particles having a concentration ranging from about 0.5% to about 5% (w/v). In one embodiment, the binding partner-enzyme conjugate is spotted on the glass fiber membrane using a solution/suspension of the binding partner-enzyme conjugate attached to the surface of particles having a concentration ranging from about 1% to about 4% (w/v). In one embodiment, the binding partner-enzyme conjugate is spotted on the glass fiber membrane using a solution/suspension of the binding partner-enzyme conjugate attached to the surface of particles having a concentration ranging from about 2% to about 3% (w/v). After the solution/suspension of the binding partner-enzyme conjugate attached to the surface of particles is applied to the glass fiber membrane, the membrane is dried, e.g., at about 37° C. for about 30 minutes.

Typically, the concentration of the binding partner-enzyme conjugate attached to the surface of particles in the glass fiber membrane ranges from 5 μg/mL to 50 μg/mL.

The thickness of the layer comprising a binding partner-enzyme conjugate attached to the surface of particles ranges from about 200 μm to about 500 μm. In one embodiment, the thickness of the layer comprising a binding partner-enzyme conjugate attached to the surface of particles ranges from about 250 μm to about 400 μm. In one embodiment, the thickness of the layer comprising a binding partner-enzyme conjugate attached to the surface of particles ranges from about 300 μm to about 400 μm. In one embodiment, the thickness of the layer comprising a binding partner-enzyme conjugate attached to the surface of particles is about 330 μm. The thickness of the layer comprising a binding partner-enzyme conjugate attached to the surface of particles depends in part on the sample volume. For example, when the sample is a very dilute solution of the analyte, the method will require a larger sample volume and a thicker layer comprising a binding partner-enzyme conjugate attached to the surface of particles will be preferred so as to accommodate the larger sample volume.

### The Read Layer

The read layer can be formed as described above.

In one embodiment, the read layer is prepared by dissolving/suspending the substrate in a solvent and applying the resulting solution/suspension to a preformed sheet of the polymer, such as a Whatman 595 filtration membrane (commercially available from GE Life Sciences, Rahway, NJ), as described above. In this embodiment, the glass fiber membrane comprising a binding partner-enzyme conjugate attached to the surface of particles is attached to the read layer by simply placing the glass fiber membrane on top of the read layer and holding the two layers held together using a frame, such as a plastic frame.

In one embodiment, the glass fiber membrane comprising a binding partner-enzyme conjugate attached to the surface of particles is attached to the read layer using a pressure sensitive adhesive between the two layers.

In one embodiment, the glass fiber membrane comprising a binding partner-enzyme conjugate attached to the surface of particles is attached to the read layer using double-sided tape. Preferably, the double-sided tape is dissolvable double-sided tape. Such a structure is depicted in FIG. 4B. In FIG. 4B the layer comprising a binding partner-enzyme conjugate attached to the surface of particles is a glass fiber membrane that is attached to a Whatman 595 read layer using dissolvable double-sided tape. In FIG. 4B the read layer is positioned on top of an optically clear PET support layer. FIG. 4C depicts a similar structure comprising a binding partner-enzyme conjugate attached to the surface of particles in a glass fiber membrane that is attached to a cellulose read layer using dissolvable double-sided tape. In FIG. 4C the cellulose read layer is coated on an optically clear PET support layer.

The double-sided tape can be selected so as to limit diffusion of the sample between each layer of the slide after the sample is applied to the layer comprising the binding partner-enzyme conjugate attached to the surface of particles. In particular, the double-sided tape prevents diffusion of the sample from the layer comprising the binding partner-enzyme conjugate attached to the surface of particles into the read layer until sufficient time has elapsed so that analyte in the sample can form a complex with the binding partner-enzyme conjugate present in the layer comprising the binding partner-enzyme conjugate attached to the surface of particles.

The time required to dissolve the intervening layer depends at least on the volume of fluid applied to it, the formulation of the intervening layer, and the thickness of the intervening layer. Dissolvable films and tapes are available. U.S. Pat. No. 7,470,397 discloses disintegrable films for diagnostic devices. U.S. Pat. No. 9,441,142 discloses adhesive tapes for use in diagnostic devices. U.S. Pat. No. 9,937,123 discloses dissolvable films for pharmaceutical or cosmetic applications. Dissolvable films and adhesives suitable for diagnostic devices are available from Adhesives Research, Inc., Glen Rock, PA. In a preferred embodiment, the intervening layer is a disintegable/dissolvable tape such as described in U.S. Published Application No. 2020/0172768, the content of which are expressly incorporated herein. In one embodiment, the intervening label is a disintegable/dissolvable tape commercially available from Adhesives Research, Inc., Glen Rock, PA.

A schematic depicting a slide comprising (i) a layer comprising a binding partner-enzyme conjugate attached to the surface of particles and (iii) a read layer, wherein the layer comprising a binding partner-enzyme conjugate attached to the surface of particles and the read layer are attached using double-sided tape, and further comprising a support layer is depicted in FIGS. 4B and 4C. In FIG. 4B the read layer is Whatman 595 membrane on a PET support layer. In FIG. 4C the read layer is a cellulose polymer coated on a PET support.

### Methods of Analysis Using the Slide

In operation, a sample suspected of containing an analyte (which can be an antigen) whose presence or amount is to be determined is added to the layer comprising a binding partner (which can be an antibody)-enzyme conjugate attached to the surface of particles, analyte (e.g., antigen) in the sample forms a complex with the binding partner-enzyme conjugate present in the layer comprising a binding partner-enzyme conjugate attached to the surface of particles. The complex of the analyte and binding partner-enzyme conjugate then diffuses into the read layer wherein the enzyme on the binding partner-enzyme conjugate (which is bound to analyte) catalyzes a reaction with the substrate to provide a signal, such as development of a color, that is measured. The signal is measured in the read layer.

The intensity of the signal is directly proportional to the concentration of the analyte in the sample, i.e., the higher the analyte concentration in the sample, the more intense the signal.

As described above, the double-sided tape can be used to limit diffusion of a sample between each layer of the slide after the sample is applied to the layer comprising a binding partner-enzyme conjugate attached to the surface of particles. In particular, the double-sided tape limits diffusion of the sample from the layer comprising the binding partner-enzyme conjugate attached to the surface of particles into the read layer until sufficient time has elapsed so that analyte in the sample can form a complex with the binding partner-enzyme conjugate.

In one embodiment, the slide further comprises one or more of a spreading layer, a reflective layer, and a carbon black-containing layer, as described below.

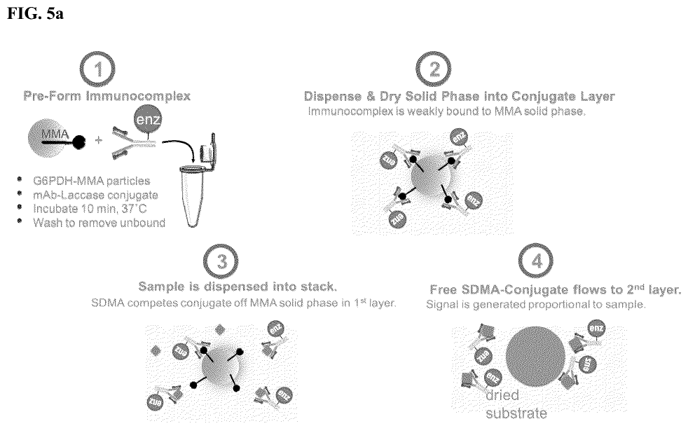

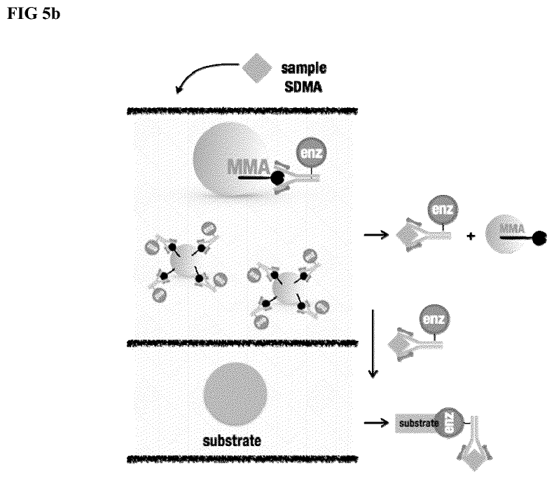

FIG. 5A illustrates the principal by which the method using a slide comprising (i) a layer comprising a binding partner-enzyme conjugate attached to the surface of particles (referred to in FIG. 5A as the “first layer” and the “conjugate layer”) and (ii) a read layer (referred to in FIG. 5A as the “second layer”). In step (1) particles with an antigen attached to their surface (G6PDH-MMA particles) are combined with an antibody-enzyme conjugate (mAB-laccase). In this illustration the antigen on the surface of the particles is mono methylarginine (MMA). The MMA is attached to the surface of particles using glucose-6-phosphate dehydrogenase (G6DPH) as a linker. The G6PDH-MMA particles and the mAB-laccase are combined, incubated for 10 minutes at 37° C., and washed to remove unbound mAB-laccase to provide antibody-enzyme conjugate attached to the surface of particles. In step (2) the resulting antibody-enzyme conjugate attached to the surface of particles are dispersed in the glass fiber membrane to provide a layer comprising a binding partner-enzyme conjugate attached to the surface of particles (i.e., the “conjugate layer”). In step (3) a sample containing SDMA (which also binds to the antibody, i.e., the mAB laccase) is applied to the conjugate layer. The SDMA competes with the MMA on the particles for binding to the mAB-laccase to provide complexes of mAb-Laccase and SDMA. In step (4) the resulting mAb-Laccase-SDMA complexes diffuse into the read layer (i.e., the second layer), wherein the laccase on the mAB reacts with substrate in the read layer to produce a signal (e.g., color change) that is proportional to the amount of SDMA in the sample. FIG. 5b is a schematic depicting the process described in FIG. 5a. FIG. 5B shows adding a sample containing SDMA to the binding partner-enzyme conjugate layer of a slide. The binding partner-enzyme conjugate layer contains particles with an antigen attached to their surface (G6PDH-MMA particles) combined with an antibody-enzyme conjugate (mAB-laccase). The G6PDH-MMA particles and the mAB-laccase are combined, incubated for 10 minutes at 37° C., and washed to remove unbound mAB-laccase to provide antibody-enzyme conjugate attached to the surface of particles. The resulting antibody-enzyme conjugate attached to the surface of particles are dispersed in a glass fiber membrane to provide a layer comprising a binding partner-enzyme conjugate attached to the surface of particles. When a sample containing SDMA is added to the binding partner-enzyme conjugate layer, the SDMA competes with the MMA on the particles for binding to the mAB-laccase to provide complexes of mAb-Laccase and SDMA. The resulting mAb-Laccase-SDMA complexes diffuse into the read layer (i.e., the second layer), wherein the laccase on the mAB reacts with substrate in the read layer to produce a signal (e.g., color change) that is proportional to the amount of SDMA in the sample.

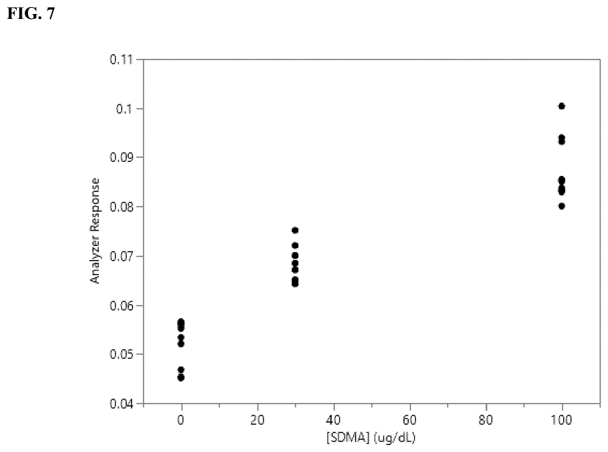

FIG. 7 depicts the results from an experiment wherein a slide having an binding partner-enzyme conjugate layer a read layer was used to assay for SDMA. FIG. 7 is a plot of Response v. SDMA concentration in a SDMA-containing sample when the sample was assayed for SDMA with a slide having an binding partner-enzyme conjugate layer a read layer. FIG. 7 shows that the response (i.e., the intensity of the signal) is proportional to the concentration of the SDMA in the sample, i.e., the higher the SDMA concentration in the sample, the more intense the response.

### 3. Assay Using a Slide Comprising (i) a Binding Partner-Enzyme Conjugate Layer, (ii) a Capture Layer, and (iii) a Read Layer

In one embodiment, the method uses a slide comprising three layers. The three layers are: (i) a binding partner-enzyme conjugate layer, (ii) a capture layer, and (iii) a read layer. The binding partner-enzyme conjugate layer comprises a binding partner that is specific for an analyte, wherein the binding partner is conjugated to (i.e., covalently bound to) an enzyme that acts as a detectable label. The capture layer comprises solid particles that have analyte molecules bound to their surface. The read layer comprises a substrate for the enzyme that produces a detectable signal (such as a color change) when the enzyme interacts with the substrate.

In one embodiment, the binding partner-enzyme conjugate layer comprises an antibody that is specific for an antigen wherein the antibody is conjugated to (i.e., covalently bound to) an enzyme that acts as a detectable label (i.e., the binding partner-enzyme conjugate layer is an antibody-enzyme conjugate layer), the capture layer comprises solid particles that have antigen molecules bound to their surface, and the read layer comprises a substrate for the enzyme that produces a detectable signal (such as a color change) when the enzyme interacts with the substrate. In this embodiment, the three layers are: (i) an antibody-enzyme conjugate layer, (ii) a capture layer, and (iii) a read layer. The slide can be used in a homogeneous enzyme immunoassay (ELISA) to determine the presence of or the amount of an antigen in a sample.

The method for determining the presence of or the amount of an analyte in a sample using the slide involves simply adding a sample suspected of containing an analyte whose presence or amount is to be determined to the binding partner-enzyme conjugate layer. The principal by which the assay works is depicted in FIG. 2 for an assay, wherein the analyte is symmetrical dimethyl arginine (SDMA) and the antibody is an antibody against SDMA.

As illustrated in FIG. 2, when the sample suspected of containing an antigen whose presence or amount is to be determined is added to the antibody-enzyme conjugate layer, antigen in the sample forms a complex with the antibody-enzyme conjugate present in the antibody-enzyme conjugate layer. The complex of the antibody and antibody-enzyme conjugate, and any un-complexed antibody-enzyme conjugate, diffuses into the capture layer where un-complexed antibody-enzyme conjugate forms a complex with antigen on the solid particles. The complex of the antibody and antibody-enzyme conjugate diffuses into the read layer wherein the enzyme on the antibody-enzyme conjugate (which is bound to antigen) catalyzes a reaction with the substrate to provide a signal, such as development of a color, that is measured. The signal is measured in the read layer.

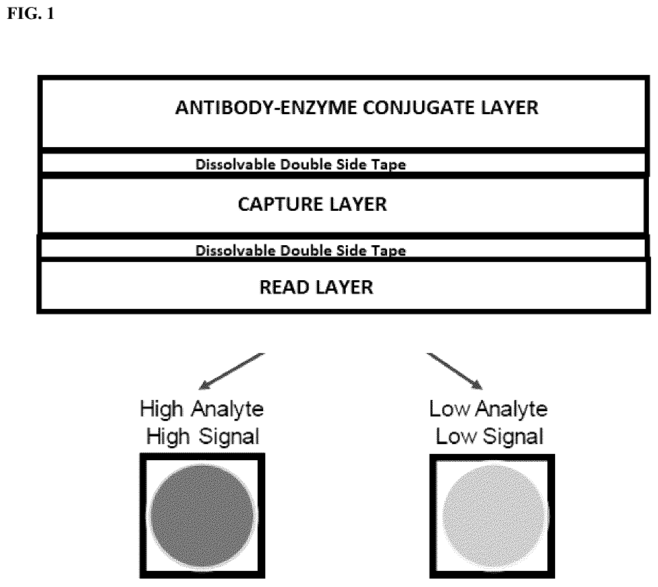

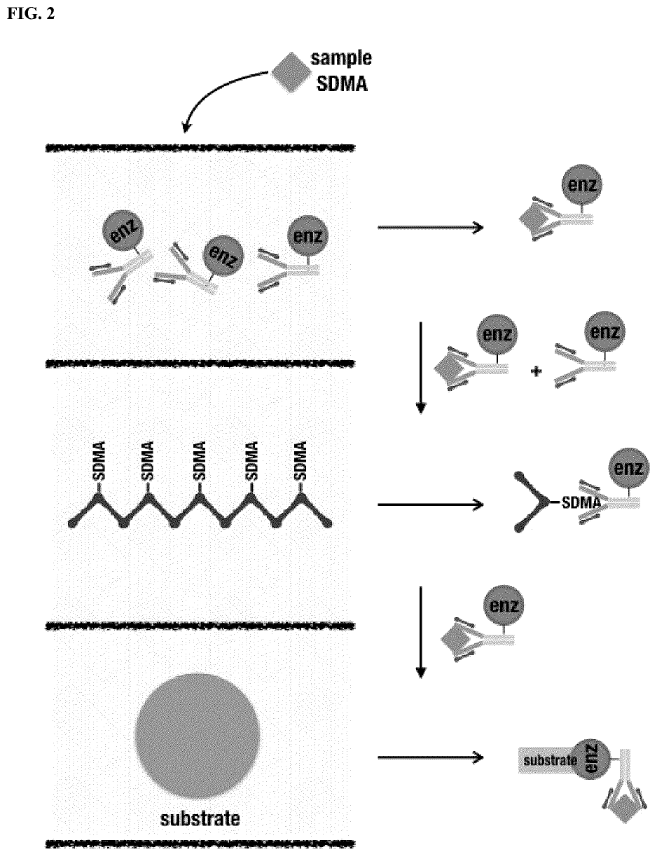

The intensity of the signal is directly proportional to the concentration of the analyte in the sample, i.e., the higher the analyte concentration in the sample, the more intense the signal, as depicted in FIG. 1. In one embodiment, there is a linear relationship between the intensity of the signal and the concentration of the analyte in the sample.

The method, advantageously, does not require any separation steps, such as washing steps.

### The Binding Partner-Enzyme Conjugate Layer

The binding partner-enzyme conjugate layer comprises a binding partner, which is specific for an analyte, that has been conjugated to an enzyme, which acts as a detectable label, so as to provide a binding partner-enzyme conjugate.

The binding partner-enzyme conjugate is dispersed in a polymer to provide the binding partner-enzyme conjugate layer. Suitable polymers for the binding partner-enzyme conjugate layer include, but are not limited to, polyesters, polypropylenes, polyethylene and polyamides. Suitable materials for the binding partner-enzyme conjugate layer preferably have a low propensity to bind to proteins.

Typically, the concentration of the binding partner-enzyme conjugate in the polymer ranges from 5 μg/mL to 50 μg/mL.

The thickness of the binding partner-enzyme conjugate layer typically ranges from about 10 μm to about 250 μm, preferably about 50 μm to about 200 μm, and more preferably about 70 μm to about 150 μm. The thickness of the binding partner-enzyme conjugate layer depends in part on the sample volume. For example, when the sample is a very dilute solution of the analyte, the method will require a larger sample volume and a thicker binding partner-enzyme conjugate layer will be preferred so as to accommodate the larger sample volume.

In one embodiment, the polymer is a polyester membrane available under the tradename Hollytex® 3254 (commercially available from Ahlstrom Munksjo, Finland). In one embodiment, the thickness of the Hollytex® 3254 is about 102 μm.

The binding partner-enzyme conjugate layer is prepared by applying a solution/suspension of the binding partner-enzyme conjugate to the polymer and then drying the polymer. In one embodiment, the solvent is an aqueous solvent. The concentration of the binding partner-enzyme conjugate in the solution/suspension ranges from about 1 μg/mL to about 10 μg/mL. In one embodiment, the concentration of the binding partner-enzyme conjugate in the solution/suspension ranges from about 2 μg/mL to about 8 μg/mL. In one embodiment, the concentration of the binding partner-enzyme conjugate in the solution/suspension is about 5 μg/mL. In one embodiment, the solvent is an aqueous solvent. In one embodiment, the solvent is phosphate buffered saline (PBS). In one embodiment, the solution/suspension containing the binding partner-enzyme conjugate further comprises polyvinylpyrrolidone and/or sucrose. In one embodiment, about 10 uL of the solution/suspension is applied to a disc of about 8 millimeter diameter of the polymer material. In one embodiment, after the solution/suspension of the binding partner-enzyme conjugate layer is applied to the polymer the polymer is dried, for example at about 40° C. for about 20 minutes.

In one embodiment, the binding partner enzyme conjugate layer is prepared by taking a polyester membrane, Hollytex® 3254 (commercially available from Ahlstrom Munksjo, Finland), and pretreating it by applying a solution of about 3% Tween-20 in PBS (a concentration of about 0.5% to about 5.0% can be used; about 0.5% is a preferred concentration) to make the membrane hydrophilic. Pretreating is accomplished by dispensing about 10 μL of the Tween-20 solution onto an about 8 mm polyester disc and then drying the disc at about 40° C. for about 20 minutes. Then about 10 μL of a solution containing the binding partner enzyme conjugate is dispensed onto the disc and the disc dried at about 40° C. for about 20 minutes. In one embodiment, the solution containing the binding partner enzyme conjugate is a solution containing the binding partner enzyme conjugate in phosphate buffered saline (PBS) at a concentration of about 5 μg/mL (range about 2 μg/mL to about 5 μg/mL), about 1% bovine serum albumin (BSA), about 0.5% sucrose, about 0.5% trehalose, about 0.1% PEG6000, about 10 nM histidine, about 10 nM tataric acid, about 10 mM Na₂B₄O₇ (sodium tetraborate), and about 0.25% Zwittergent® 3-14 (commercially available from MilliporeSigma, Burlington, MA). The histidine is optional and is used to control the pH. Other amino acids, such as serine and glycine, can be used in place of histidine. The Zwittergent® 3-14 can be replaced with Igepal® CA-630 (commercially available from Sigma Aldrich, St. Louis, MO) and Tween-20. Other buffers, such as citrate-phosphate buffer can be used in place of PBS. In one embodiment, the binding partner enzyme conjugate is laccase conjugated to an anti-SDMA antibody.

Methods for forming a binding partner-enzyme conjugate are discussed above.

Illustrative enzymes are described above.

### The Capture Layer

The capture layer comprises solid particles having analyte molecules attached to the surface thereof. The solid particles with the attached analyte molecules are dispersed in a polymer to provide the capture layer. The solid particles with the attached analyte molecules are entrapped in the polymer and cannot diffuse into other layers of the slide.

Solid particles to which analyte molecules can be attached are described above.

Methods for attaching analyte particles to solid particles are described above.

Suitable polymers for the capture layer include, but are not limited to, polycarbonates. In one embodiment, the capture layer is a polycarbonate polymer having a pore size of about 0.8 μm. In one embodiment, the polymer is a polycarbonate track etched (PCTE) membrane, 0.8 μm pore size (commercially available from GE Life Sciences, Piscataway, NJ).

Typically, the amount of the solid particles with the attached analyte molecules applied to the polymer ranges from 5 uL of a 1% (w/v) suspension of particles, to 50 uL of a 1% (w/v) suspension of particles.

The thickness of the capture layer ranges from about 2 μm to about 20 μm, preferably about 5 μm to about 15 μm in diameter, and more preferably about 7 μm to about 12 μm in diameter. The thickness of the capture layer depends in part on the sample volume. For example, when the sample is a very dilute solution of the analyte, the method will require a larger sample volume and a thicker capture layer will be preferred so as to accommodate the larger sample volume.

The capture layer is prepared by applying a solution/suspension of the solid particles with the analyte attached to their surface to the polymer and then drying the polymer. In one embodiment, the solvent is an aqueous solvent. The concentration of the solid particles with the analyte attached to their surface in the solution/suspension ranges from about 0.2% to about 2.0%. After the solution/suspension of the analyte attached to their surface to the polymer is applied to the polymer the polymer is dried, e.g., at about 37° C. for about 20 minutes.

In one embodiment, the capture layer comprises solid particles having antigen molecules attached to the surface thereof. The solid particles with the attached antigen molecules are dispersed in the polymer to provide the capture layer.

An embodiment encompasses the solid particles with antigen attached to their surface.

An embodiment encompasses the solid particles with antigen attached to their surface dispersed in a polymer.

In one embodiment, the solid particles with antigen attached to their surface dispersed in a polymer are latex particles, about 1 μm to about 2 μm, passively coated with SDMA attached to the surface of particles using glucose-6-phosphate dehydrogenase (G6DPH) as a linker (i.e., latex particles coated with G6DPH-SDMA). In one embodiment, the polymer is a polycarbonate track etched (PCTE) membrane, 0.8 μm pore size (commercially available from GE Life Sciences, Piscataway, NJ). In one embodiment, a Nordson EFD dispenser (commercially available from Nordson EFD, East Providence, RI) is used to apply about 18.5 uL of the G6DPH-SDMA particles (1% w/v particles in 50 mM phosphate buffer pH 7.3, 2.5% sucrose) to the polymer.

In another embodiment, the solid particles with antigen attached to their surface dispersed in a polymer are latex particles, about 1 μm to about 2 μm, passively coated with MMA attached to the surface of particles using glucose-6-phosphate dehydrogenase (G6DPH) as a linker (i.e., latex particles coated with G6DPH-MMA).

### The Read Layer

The read layer can be formed as described above.

### Optional Intervening Layers

In one embodiment, the layers are separated by an intervening layer, such as, for example, double-sided tape. In one embodiment, at least the binding partner-enzyme conjugate layer is separated from the capture layer by an intervening layer. In one embodiment, at least the capture layer is separated from the read layer by an intervening layer. In one embodiment, the binding partner-enzyme conjugate layer is separated from the capture layer by an intervening layer and the capture layer is separated from the read layer by an intervening layer.

The intervening layers are selected so as to limit diffusion of a sample between each layer of the slide after the sample is applied to the binding partner-enzyme conjugate layer. In particular, the intervening layer between the binding partner-enzyme conjugate layer and the capture layer prevents diffusion of the sample from the binding partner-enzyme conjugate layer into the capture layer until sufficient time has elapsed so that analyte in the sample can form a complex with the binding partner-enzyme conjugate present in the binding partner-enzyme conjugate layer. Similarly, the intervening layer between the capture layer and the read layer prevents diffusion of the sample from the capture layer into the read layer until sufficient time has elapsed so that binding partner-enzyme conjugate that has not formed a complex with analyte in the sample can form a complex with analyte on the solid particles.

The general structure of the slide, including intervening layers, for determining the amount of an antigen in a sample is depicted in FIG. 1.

In one embodiment, the intervening layer is a double-sided tape. Typically, the thickness of the double-sided tape ranges from about 10 μm to about 100 μm, preferably from about 15 μm to about 90 μm, and more preferably from about 20 μm to about 80 μm. In one embodiment the thickness of the double-sided tape is about 60 μm.

Initially, the double-sided tape is water impermeable but, with prolonged exposure to water, the tape dissolves. The time required for the tape to dissolve depends on the formulation of the tape. Dissolution times can vary from seconds to minutes. In one embodiment, the dissolution time of the tape ranges from about 1 minute to about 5 minutes. In one embodiment, the dissolution time of the tape ranges from about 2 minutes to about 4 minutes. Suitable double-sided tape is described above.

In one embodiment, the sample is applied to the slide more than once. In a preferred embodiment, the sample is applied to the slide twice, each application being separated by a time period, t. In one embodiment, the time period, t, ranges from about 5 seconds to about 10 minutes. In one embodiment, the time period, t, ranges from about 1 minute to about 5 minutes. In one embodiment, the time period, t, ranges from about 2 minutes to about 3 minutes. In one embodiment, the amount of sample applied during each application ranges from about 1 uL to about 100 uL. In one embodiment, the amount of sample applied during each application ranges from about 2 uL to about 50 uL. In one embodiment, the amount of sample applied during each application ranges from about 3 uL to about 25 uL. In one embodiment, the amount of sample applied during each application ranges from about 5 uL to about 15 uL. In one embodiment, the amount of sample applied during each application ranges from about 6 uL to about 11 uL.

Without wishing to be bound by theory, it is believed that a single application of the sample is insufficient to dissolve the double-sided tape. Application of the sample a second time, however, is sufficient to dissolve the tape. Thus, by varying the time between first application of the sample and the second application of the sample, it is possible to control, for example, the time for analyte in the sample to form a complex with the binding partner-enzyme conjugate present in the binding partner-enzyme conjugate layer before the sample from the binding partner-enzyme conjugate layer diffuses into the capture layer. In one embodiment, the intervening layer is a disintegable/dissolvable tape such as described in U.S. Published Application No. 2020/0172768, the content of which are expressly incorporated herein. In one embodiment, the intervening label is a disintegable/dissolvable tape commercially available from Adhesives Research, Inc., Glen Rock, PA.

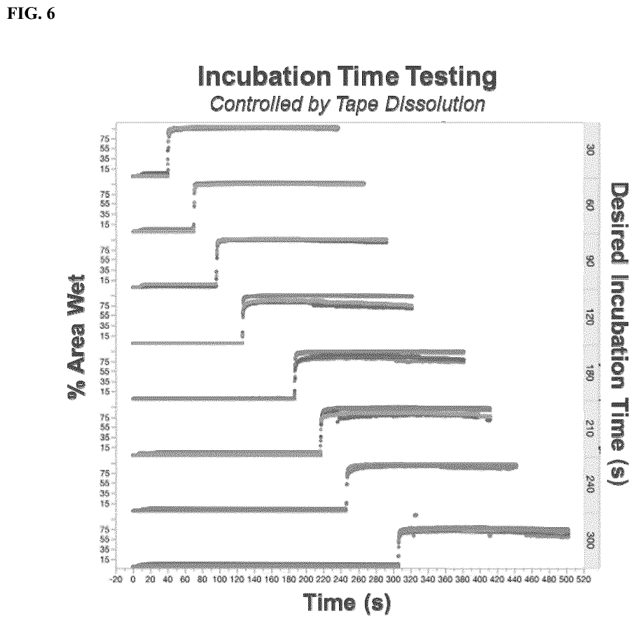

FIG. 6 is a plot of the percent area of a read layer that is wetted when a first aqueous sample is applied to a slide having a binding partner-enzyme conjugate layer and a read layer that are separated by dissolvable double-sided tape followed by application of a second aqueous sample after a period of time, t, between application of the first aqueous sample and the second aqueous sample. The read layer darkens when it is wetted. Wetness of the read layer was assessed using a camera. FIG. 6 shows that the first aqueous sample is insufficient to dissolve the tape and allow the water to reach the read layer. However, application of the second aqueous sample after the time period t provides complete wetting of the read layer almost immediately. Using dissolvable double-sided tape allows careful control of how long a sample containing an analyte will remain in the binding partner-enzyme conjugate layer before it comes in contact with the read layer.

### Other Optional Layers

The slide can optionally include one or more of a spreading layer, a reflective layer, and a carbon black layer.

### The Spreading Layer

In one embodiment, the slide further comprises a spreading layer. The spreading layer is coated on top of the binding partner-enzyme conjugate layer. The spreading layer is water permeable. The spreading layer is water permeable and is isotropically porous, i.e., it is porous within every direction within the layer.

The spreading layer is provided by combining the components of the layer in a solvent with mixing to provide a solution/suspension, the solution/suspension applied to the binding partner-enzyme conjugate layer, and the solvent removed so as to provide the spreading layer on top of the binding partner-enzyme conjugate layer.

The thickness of the wet spreading layer typically ranges from about 100 μm to about 500 μm, preferably about 150 μm to about 450 μm, more preferably about 200 μm to about 400 μm, and most preferably about 250 μm to about 350 μm. When dry, the thickness of the spreading layer typically ranges from about 25 μm to about 250 μm, preferably about 50 μm to about 200 μm, and more preferably about 75 μm to about 150 μm. The thickness of the spreading layer depends in part on the sample volume. For example, when the sample is a very dilute solution of the analyte, the assay will require a larger sample volume and a thicker spreading layer will be preferred so as to accommodate the larger sample volume.

The spreading layer is typically a mixture of cellulose in a hydrophilic polymer matrix. Suitable hydrophilic polymers include, but are not limited to, polyacrylic acid, polyvinylpyrrolidone, polyethylene glycol, polyethylene oxide, polyvinyl alcohol, polyacrylamides, and polyethyleneimines. The spreading layer advantageously spreads and disperses the liquid sample evenly over the slide.

The amount of cellulose in the wet spreading layer typically ranges from about 1% by wt. to about 20% by wt., preferably 5% by wt. to about 15% by wt., and more preferably about 10% by wt. of the layer. When dry, the amount of cellulose in the dry spreading layer typically ranges from about 50% by wt. to about 98% by wt., preferably about 60% by wt. to about 95% by wt., and more preferably about 70% by wt. to about 90% by wt. of the layer. In one embodiment, the spreading layer is 100% cellulose. In one embodiment, the cellulose has a particle size of about 300 μm.

The spreading layer may include polyvinylpyrrolidone (PVP). The amount of PVP by weight in the wet spreading layer typically ranges from about 0.5% by wt. to about 10% by wt., preferably about 1% by wt. to about 5% by wt., more preferably about 1.5% by wt. to about 3% by wt. of the layer. In one embodiment, the amount of PVP in the wet spreading layer is about 2% by wt. The amount of PVP by weight in the dry spreading layer typically ranges from about 1% by wt. to about 30% by wt., preferably about 5% by wt. to about 25% by wt., more preferably about 10% by wt. to about 20% by wt. of the layer. In one embodiment, the amount of PVP in the dry spreading layer is about 17% by wt.

The spreading layer may include tetramethylammonium hydroxide (TMAH) or other suitable base. The TMAH may serve to adjust the pH of the spreading layer. Without wishing to be bound by theory, it is believed that TMAH increases the ability of the spreading layer to absorb and spread the sample rapidly across the whole slide. The amount of TMAH by weight in the wet spreading layer typically ranges from about 0.01% by wt. to about 0.5% by wt., preferably about 0.025% by wt. to about 0.25% by wt. In one embodiment, the amount of TMAH in the wet spreading layer is about 0.05% by wt. The amount of TMAH by weight in the dry spreading layer typically ranges from about 0.08% by wt. to about 4% by wt., preferably about 0.2% by wt. to about 2% by wt., more preferably about 10% by wt. In one embodiment, the amount of TMAH in the dry spreading layer is about 0.4% by wt.

In one embodiment, the spreading layer is formed by applying a solution/suspension containing about 10% by wt. of cellulose, about 2% by wt. of polyvinylpyrrolidone (PVP), about 68% by wt. of water, about 20% by wt. of ethanol, about 0.06% by wt. of polyacrylic acid (PAA), and about 0.05% by wt. of tetramethylammonium hydroxide (TMAH) and removing solvent from the solution/suspension. The resulting dry spreading layer contains about 82.6% by wt. of cellulose, about 16.5% by wt. of PVP, about 0.5% by wt. of PAA, and about 0.4% by wt. of TMAH.

### The Reflective Layer

In one embodiment, the slide further comprises a reflective layer. The reflective layer is coated on top of the binding partner-enzyme conjugate layer. In one embodiment, the slide further comprises a spreading layer and a reflective layer. When the slide comprises both a spreading layer and a reflective layer, the reflective layer is positioned between the spreading layer and the binding partner-enzyme conjugate layer.

The reflective layer is provided by combining the components of the layer in a solvent with mixing to provide a solution/suspension, the solution/suspension applied to the binding partner-enzyme conjugate layer, and the solvent removed so as to provide the spreading layer on top of the binding partner-enzyme conjugate layer.

In one embodiment, the reflective layer comprises titanium oxide (TiO₂). Typically, the titanium dioxide is present in the wet reflective layer in an amount ranging from about 2% by wt. to about 30% by wt., preferably about 5% by wt. to about 20% by wt., and more preferably about 10% by wt. to about 20% by wt. of the layer. In one embodiment, the titanium oxide is present in the wet reflective layer in an amount of about 14% by wt. of the layer. Typically, the titanium dioxide is present in the dry reflective layer in an amount ranging from about 20% by wt. to about 60% by wt., preferably about 25% by wt. to about 55% by wt., and more preferably about 30% by wt. to about 50% by wt. of the layer. In one embodiment, the titanium oxide is present in the wet reflective layer in an amount of about 42% by wt. of the layer.

The average particle size of the titanium oxide particles is typically less than about 10 μm and preferably less than about 5 μm. In one embodiment, the average particle size of the titanium oxide particles is about 0.35 μm.

The titanium oxide can be replaced with, or used in combination with, other reflective materials. Illustrative other reflective materials include, but are not limited to barium sulfate, zinc oxide, clay, and aluminum silicate. In one embodiment, the reflective material is fully or partially metal-coated particles. Suitable metals include, but are not limited, to aluminum, gold, nickel or silver. Silver is a preferred coating. These particles can be used alone or in combination with other light reflective materials such as TiO₂. The particles (i.e., the core that is coated) can be a variety of materials, including, but not limited to, solid and hollow glass, poly(methyl methacrylate) (PMMA), and silica. Suitable metal-coated particles are commercially available, e.g., from Cospheric LLC (Santa Barbara, California, USA). Preferred particles are silver-coated silica microspheres, for example from Cospheric LLC.

In one embodiment, the layer is formed by applying a solution/suspension containing about 7% by wt. of cellulose, about 14% by wt. of titanium dioxide, and about 79% by wt. of a D4 hydrogel solution (containing about 16% by wt. of a mixture of equal amounts of low viscosity HydroMed D4 and high viscosity HydroMed D4), about 90% by wt. of ethanol, and about 10% by wt. of water) and removing solvent from the solution/suspension. The resulting dry filtering layer contains about 20% by wt. of cellulose, about 42% by wt. of titanium dioxide, and about 38% by wt. of HydroMed D4. HydroMed D4 is commercially available from AdvanSource Biomaterials Corp of Wilmington, MA.

A reflective layer is particularly advantageous when the slide includes a carbon black layer, as described below, because the carbon black layer absorbs scattered light. The reflective layer advantageously reflects the light away from the carbon black layer and back towards the detector, so as to prevent the carbon black layer from absorbing the scattered light, resulting in improved sensitivity.

### The Carbon Black Layer

In one embodiment, the slide further comprises a carbon black layer. The carbon black layer is coated on top of the reflective layer and positioned between the reflective layer and the spreading layer. The carbon black layer acts as an optical barrier to advantageously filter stray light from the environment that interferes with the measuring the intensity of the signal. The carbon black layer can also function to bind compounds present in the sample other than the analyte that could potentially interfere with the assay and prevents these other compounds from diffusing into the read layer.

The carbon black can be replaced with, or used in combination with, other materials to filter stray light. Illustrative other materials include, but are not limited to black latex beads, black silica beads, activated charcoal, C₆₀, graphene, or other colored materials such as red latex beads.

The carbon black layer comprises carbon black dispersed in a polymer. Suitable polymers for the carbon black layer include, but are not limited to, polyurethane (such as HydroMed D4, (commercially available from AdvanSource Biomaterials Corp of Wilmington, MA), polyethylene oxide, silicones, polyvinyl alcohol, and polyacrylamides. In one embodiment, the polymer is HydroMed D4.

The carbon black layer is provided by combining the components of the layer in a solvent with mixing to provide a solution/suspension, the solution/suspension applied to the reflective layer (or, if no reflective layer, the binding partner-enzyme conjugate layer), and the solvent removed so as to provide the carbon black layer on top of the reflective layer.

The amount of carbon black in the wet carbon black layer typically ranges from about 1% by wt. to about 20% by wt., preferably about 1.5% by wt. to about 15% by wt., more preferably about 2% by wt. to about 10% by wt. of the layer. In one embodiment, the amount of carbon black in the wet carbon black layer is about 5% by wt. of the layer. When dry, the amount of carbon black in the carbon black layer typically ranges from about 10% by wt. to about 40% by wt., preferably about 15% by wt. to about 35% by wt., more preferably about 20% by wt. to about 30% by wt. of the layer. In one embodiment, the amount of carbon black in the wet carbon black layer is about 25% by wt. of the layer.

The thickness of the wet carbon black layer typically ranges from about 50 μm to about 300 μm, preferably about 75 μm to about 250 μm, and more preferably about 100 μm to about 200 μm. In one embodiment, the thickness of the wet carbon black is about 140 μm. When dry, the thickness of the carbon black layer typically ranges from about 5 μm to about 30 μm, preferably about 7.5 μm to about 25 μm, and more preferably about 10 μm to about 20 μm. In one embodiment, the thickness of the wet carbon black is about 14 μm.

In one embodiment, the carbon black layer is formed by applying a solution/suspension containing about 4.7% by wt. of carbon black, about 95.3% by wt. of a D4 hydrogel solution (containing about 16% by wt. of a mixture of equal amounts of low viscosity HydroMed D4 and high viscosity HydroMed D4, about 90% by wt. of ethanol, and about 10% by wt. of water) and removing solvent from the solution/suspension. The resulting dry carbon black layer contains about 24% by wt. of carbon black and about 76% by wt. of HydroMed D4.

As noted above, when the slide includes a carbon black layer the slide preferably also includes a reflective layer.

A description of these optional layers and how they are formed is described in co-pending U.S. Published Application No. 2022/0082501.

Typically, the total thickness of the slide is less than 550 μm, preferably less than 500 μm.

In one embodiment, the slide is part of a device wherein the liquid sample containing the analyte of interest is applied to an aperture on the device and the liquid then conveyed to the dry slide via a capillary transport zone. Illustrative examples of such a device are described in U.S. Pat. Nos. 4,323,536 and 5,726,010.

### 4. General Features of the Methods

The molecular weight of the analyte can vary over a wide range. Typically, the molecular weight of the analyte is greater than 50 Daltons. For analytes that are small molecules, the molecular weight of the analyte is typically between about 50 and about 4,000 Daltons, preferably about 100 to about 2,000 Daltons. When the analyte is a larger molecule, such as a protein, the molecular weight can be greater than 2,000 Daltons. When the analyte is a larger molecule, such as a protein, the molecular weight can even be greater than 4,000 Daltons. The method works well for analytes that are large molecules (e.g., molecular weights over 2000 Daltons), such as proteins.

The method can be used to assay for a wide variety of analytes. Illustrative analytes include, but are not limited to, small molecules (e.g., symmetrical dimethyl arginine (SDMA), asymmetrical dimethyl arginine (ADMA), mono methylarginine (MMA), melamine, antibiotics, T4, β-lactam antibiotics (such as penicillin), sulfa drugs, cephalosporins, and steroids (e.g., progesterone and cortisol)), beta aminoisobutyric acid, cystatins, fibroblast growth factors, and clusterin.

In a preferred embodiment, the analyte is an antigen and the binding partner is an antibody that is specific for the antigen.

In one embodiment, the analyte is SDMA. The structure of SDMA is:

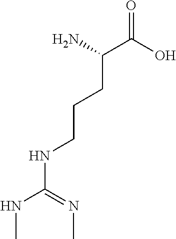

In one embodiment, the analyte is melamine.

In one embodiment, the analyte is T4.

In one embodiment, the analyte is cortisol.

In one embodiment, the analyte is progesterone.

In one embodiment, the analyte is cystatin-B.

In one embodiment, the analyte is mono methyl arginine.

In one embodiment, the analyte is a fibroblast growth factor.

In one embodiment, the analyte is an antibiotic. Illustrative antibiotics include, but are not limited to, amoxicillin, ampicillin, cefacetrile, cefquinome, cefazolin, cefoperazone, ceftiofur, cephalexin, cefalonium, cloxacillin, desacetyl cephapirin, dicloxacillin, nafcillin, oxacillin, cephapirin, desfuroylceftiofur, cefuroxime, and penicillin.

In one embodiment, the analyte is one or more antibiotics selected from the group consisting of the antibiotics that must be tested for in milk as required by the European Union.

In one embodiment, the analyte is one or more antibiotics selected from the group consisting of penicillin G (benzylpenicillin), ampicillin, amoxicillin, oxacillin, cloxacillin, dicloxacillin, nafcillin, cephapirin, desacetylcephapirin, ceftiofur, desfuroylceftiofur, cefquinome, cefalonium, cefazolin, cefacetrile, cephalexin, cefuroxime, and cefoperazone

In one embodiment, the analyte is one or more antibiotics selected from the group consisting of the antibiotics that must be tested for in milk as required by the United States Food and Drug Administration.

In one embodiment, the analyte is one or more antibiotics selected from the group consisting of penicillin G (benzylpenicillin), ampicillin, amoxicillin, cloxacillin, cephapirin, ceftiofur, and desfuroylceftiofur.

In one embodiment, the analyte is a peptide and the binding partner is a receptor that is specific for the peptide.

In one embodiment, the analyte is a penicillin and the binding partner is a penicillin binding protein.

In one embodiment, the analyte is a steroid and the binding partner is a steroid binding protein that is specific for the steroid.

In one embodiment, the sample is an aqueous solution.

In one embodiment the sample is urine.

In one embodiment, the sample is serum.

In one embodiment, the sample is milk.

In one embodiment, the sample is saliva.

In one embodiment, the sample is plasma.

In one embodiment, the sample is whole blood.

In one embodiment, the sample is sweat.

In one embodiment, the sample is tears.

In one embodiment, the sample is spinal fluid.

In one embodiment, the sample is a fecal sample or fecal extract.

In one embodiment, the sample is obtained from a mammal.

Typically, the sample size ranges from about 1 uL to about 100 uL.

The concentration of the analyte in the sample can vary over a wide range. Generally, the concentration of the analyte in the sample ranges from about 1 ng/mL to about 400 ng/mL. One will readily understand that more concentrated samples can simply be diluted with an appropriate diluent to provide a diluted sample having a concentration that is suitable for the assay. The method is extremely sensitive and for some analytes can be used to detect the analyte at a concentration as low as 1 ng/mL.

### EXAMPLES

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1: Conjugation of Laccase to Anti-SDMA Antibody

The following method can be used to conjugate laccase to an anti-SDMA antibody.

### Step #1: Capping of Laccase Free Thiols

To a 3 mL solution of laccase (9.1 mg/mL in PBS (phosphate buffered saline), pH 7.4, commercially available from Sigma-Aldrich and purified using a ConA column (Commercially available from ThermoFisher Scientific) was added 36 μL of iodoacetamide solution (100 mg/mL in DMSO). The resulting solution was covered with foil and rotated end over end at room temperature for 1 hour. The capped laccase was dialyzed in PBS, pH 7.4 at 4° C., exchanging three times with 4 L of PBS. After dialysis, the concentration of laccase was determined by measuring the absorbance at 280 nm (coefficient of Abs: 2.1=1.0 mg/mL). The concentration was determined to be 9.0 mg/mL.

Step #2: Activation of Laccase with SMCC

To a 3 mL solution of capped laccase at a concentration 9.0 mg/mL was added 76 μL of sulfo-SMCC (sulfosuccinimidyl 4-(N-maleimidomethyl)cyclohexane-1-carboxylate) (Commercially available from Sigma-Aldrich) solution (20 mg/mL in DMSO). The resulting solution was rotated end over end at room temperature for 1 hour. The activated laccase was dialyzed in PBS, pH 7.4 at 4° C., exchanging three times with 4 L of PBS. After dialysis, the concentration of laccase was determined by measuring the absorbance at 280 nm (coefficient of Abs: 2.1=1.0 mg/mL). The concentration was determined to be 8.5 mg/mL.

### Step #3: Reduction of Anti-SDMA Antibody by DTT

To a 3 mL solution of anti-SDMA antibody (5.4 mg/mL in PBS, pH 7.4, purchased from Leinco Technologies of Fenton, MO) was added 30 μL of 0.5M EDTA solution and 12 μL of DTT (dithiothreitol) solution (500 mM in deionized H₂O, no weigh format). The resulting solution was rotated end over end at room temperature for 1 hour. The resulting solution was purified using a desalting size-exclusion PD-10 column) (commercially available from Sigma-Aldrich), using PBS, pH 7.4+5 mM EDTA as eluent. Fractions were collected in 0.5 mL aliquots. Presence of protein was determine using absorbance at 280 nm. Fractions containing protein were combined and the concentration was determined by measuring absorbance at 280 nm (coefficient of Abs: 1.37=1.0 mg/mL). The concentration was determined to be 2.5 mg/mL.

### Step #4: Conjugation of Laccase to Anti-SDMA Antibody

To 2 mL of reduced anti-SDMA antibody at 2.5 mg/mL in PBS+5 mM EDTA was added 2.4 mL of activated laccase in PBS at 8.5 mg/mL. The solution was rotated end over end for 18 hours at 4° C. Then, 2 μL of a 0.5 M L-cysteine solution in deionized H₂O was added to quench the reaction. The resulting solution of laccase conjugate to anti-SDMA antibody was rotated end over end for 30 minutes and then stored at 4° C. until further use.

### Example 2: Coating of the Laccase Anti-SDMA Conjugate onto MMA-Coated Particles

The following method was used to coat the laccase anti-SDMA conjugate onto MMA-coated particles:

Materials
1. Laccase-Anti-SDMA Conjugate, 1.33 mg/mL Anti-SDMA,
2. G6PDH-MMA, 12.5 eq, 1.0 μm, pH 5.2 MES buffer
3. Calibrator Diluent, 1% BSA, 200 mM phosphate buffer, 0.1% Proclin, pH=7.4,
4. 1× Wash Buffer, 0.1% Tween in DI H₂O
5. Serum calibrators, Canine Charcoal Stripped, 0.2 μm filtered, 0 ug/dL, 7 ug/dL, 14 ug/dL, 30 ug/dL, 100 ug/dL,
6. TMB solution −0.5 ug/mL TMB, 50 mM succinic, 1:1 PBS, pH=3.5.

The following procedure was used to form an immunocomplex between the laccase-anti-SDMA conjugate described above and the latex particles coated with MMA-G6PDH:
1. Placed 746 uL of 1.34% G6PDH-MMA particles in centrifuge tube;
2. Centrifuged at 13,300 rpm for 2 minutes, removed supernatant;
3. Added 440 uL of calibrator diluent, resuspended particles by pipetting;
4. Sonicated for 1 minute;
5. Added 60 uL of Laccase-Anti-SDMA conjugate (total solids=2%), incubated 10 min at 37° C.;
6. Centrifuged 13,300 rpm for 2 minutes, removed supernatant;
7. Washed with 500 uL of 1× Wash, centrifuged as above and removed supernatant;
8. Repeated wash in Step 7 two more times.

A Competition Assay Protocol was used to confirm that the immunocomplex particles had been successfully formed and were active, as follows:
1. Added 500 uL calibrator diluent to particles, resuspended, sonicated for 2 minute (2% solids);
2. Distributed 50 uL of complexed particles into tubes, centrifuged as above, removed supernatant;
3. Added 50 uL of sample containing different levels of SDMA. Resuspended particles, vortexed briefly;
4. Incubated at 37° C. for 90 seconds, centrifuged as above;
5. Plated 25 uL of sample into 96-well plate;
6. Added 175 uL TMB solution, read at 645 nm at 37° C. for 15 minutes.

The entire disclosure of all references that have been cited are incorporated herein by reference.

What is claimed is:

1. A slide for determining the presence of or the amount of an analyte in a sample comprising:
(i) a first layer comprising a binding partner-enzyme conjugate dispersed in a first polymer, wherein the binding partner-enzyme conjugate is specific for the analyte;
(ii) a second layer comprising solid particles that have analyte bound to their surface dispersed in a second polymer, and
(iii) a third layer comprising a substrate for the enzyme dispersed in a third polymer, wherein interaction of the substrate and the enzyme produces a detectable signal.

2. The slide of claim 1, wherein the binding partner is an antibody.

3. The slide of claim 1 further comprising an intervening layer between at least one of the first layer and the second layer or the second layer and the third layer, wherein the intervening layer limits diffusion of a sample between each layer.

4. The slide of claim 1 wherein the first layer is a polyester membrane, wherein the thickness of the polyester membrane is about 100 μm.

5. The slide of claim 1, wherein the concentration of the binding partner-enzyme conjugate dispersed in the first polymer ranges from about 5 μg/mL to about 50 μg/mL.

6. The slide of claim 1, wherein the enzyme in the binding partner-enzyme conjugate is selected from the group consisting of a laccase, a beta-galactosidase, a beta-lactamase, and a luciferase.

7. The slide of claim 1, wherein the enzyme in the binding partner-enzyme conjugate is a laccase selected from the group consisting of Myceliophthora thermophila, Thermothelomyces thermophila, Botrytis aclada, Streptomyces cyanes, and Thermus thermophilus.

8. The slide of claim 1, wherein the second polymer is a polycarbonate track etched (PCTE) membrane having a 0.8 μm pore size a polycarbonate.

9. The slide of claim 1, wherein the solid particles are latex particles of about 0.5 μm to about 5.0 μm in diameter.

10. The slide of claim 1, wherein the concentration of the solid particles that have analyte bound to their surface in the second polymer ranges from about 5% by wt to about 30% by wt.

11. The slide of claim 1, wherein the thickness of the second layer ranges from about 2 μm to about 20 μm.

12. The slide of claim 1, wherein the third layer is a cellulose polymer.

13. The slide of claim 1, wherein the concentration of the substrate in the third polymer ranges from about 0.1% to about 5% by wt.

14. The slide of claim 1, wherein the thickness of the third layer ranges from about 25 μm to about 300 μm.

15. The slide of claim 1, wherein the wherein the enzyme in the binding partner-enzyme conjugate is a laccase and the substrate is 3,3′,5,5′-tetramethylbenzidine (TMB) present as an ion-pair formed between TMB and a divalent acid.

16. The slide of claim 15, wherein the divalent acid is selected from the group consisting of succinic acid, oxalic acid, malonic acid, glutamic acid, adipic acid, and pimelic acid.

17. The slide of claim 16, wherein the divalent acid is succinic acid.

18. A slide for determining the presence of or the amount of an analyte in a sample comprising:
(i) a first layer comprising a binding partner-enzyme conjugate attached to the surface of particles dispersed in a first polymer, wherein the binding partner-enzyme conjugate is specific for the analyte;
(ii) a second layer comprising a substrate for the enzyme dispersed in a second polymer, wherein interaction of the substrate and the enzyme produces a detectable signal.

19. The slide of claim 18, wherein the binding partner is an antibody.

20. The slide of claim 18, further comprising an intervening layer between the first layer and the second layer, wherein the intervening layer limits diffusion of a sample between each layer.

21. The slide of claim 18, wherein the first layer is a glass fiber membrane.

22. The slide of claim 18, wherein the concentration of the binding partner-enzyme conjugate attached to the surface of particles in the first polymer ranges from about 5 μg/mL to about 50 μg/mL.

23. The slide of claim 18, wherein the thickness of the first layer ranges from about 50 μm to about 500 μm.

24. The slide of claim 18, wherein the second layer is a cellulose polymer.

25. The slide of claim 18, wherein the concentration of the substrate in the second polymer ranges from about 0.1% to about 5% by wt.

26. The slide of claim 18, wherein the thickness of the second layer ranges from about 25 μm to about 300 μm.

27. The slide of claim 18, wherein the wherein the enzyme in the binding partner-enzyme conjugate is a laccase and the substrate is 3,3′,5,5′-tetramethylbenzidine (TMB) present as an ion-pair formed between TMB and a divalent acid.

28. The slide of claim 27, wherein the divalent acid is selected from the group consisting of succinic acid, oxalic acid, malonic acid, glutamic acid, adipic acid, and pimelic acid.

29. The slide of claim 28, wherein the divalent acid is succinic acid.

30. A method for determining the presence of or the amount of an analyte in a sample comprising:
(i) providing a sample suspected of containing an analyte;
(ii) contacting the sample with the first layer of the slide of claim 1; and
(iii) determining if there is a detectable signal.

31. The method of claim 30, further comprising measuring the intensity of the detectable signal.

32. A method for determining the presence of or the amount of an analyte in a sample comprising:
(i) providing a sample suspected of containing an analyte;
(ii) contacting the sample with the first layer of the slide of claim 1; and
(iii) determining if there is a detectable signal.

33. The method of claim 32, further comprising measuring the intensity of the detectable signal.

34. A method for determining the presence of or the amount of an analyte in a sample comprising:
(i) providing a sample suspected of containing an analyte;
(ii) contacting the sample with a binding partner-enzyme conjugate attached to the surface of particles to provide a mixture of comprising the binding partner-enzyme conjugate attached to the surface of particles and the sample, wherein analyte in the mixture can displace the binding partner-enzyme conjugate from the surface of the particles,
(iii) separating the particles from the mixture to provide a particle free mixture;
(iv) contacting the particle free mixture with a polymer comprising a substrate for the enzyme dispersed in the polymer, wherein interaction of the substrate and the enzyme produces a detectable signal.

35. A dry slide for determining the presence of or the amount of an analyte in a sample comprising dried laccase.

36. The slide of claim 35, wherein the laccase is conjugated to an antibody specific for the analyte.

37. The slide of claim 36, further comprising TMB and succinic acid.

38. A dry slide for determining the presence of or the amount of an analyte in a sample comprising TMB and succinic acid.
